(19) United States
(12) Patent Application Publication
(10) Pub. No.: US 2026/0130841 A1
(43) Pub. Date: May 14, 2026

(54) SKIN EXTERNAL COMPOSITION COMPRISING CROSSLINKED POLYGLUTAMIC ACID

(71) Applicant: Amorepacific Corporation, Seoul (KR)

(72) Inventors: Young Suk CHO, Yongin-si (KR); Young Gyu KANG, Yongin-si (KR); Da Jeong LEE, Yongin-si (KR); Jeong Yu LEE, Yongin-si (KR)

(73) Assignee: Amorepacific Corporation, Seoul (KR)

(21) Appl. No.: 19/387,780

(22) Filed: Nov. 13, 2025

(30) Foreign Application Priority Data
Nov. 14, 2024 (KR) 10-2024-0161678

(51) Int. Cl. A61K 8/88; A61K 8/06; A61Q 19/00

(52) U.S. Cl. CPC A61K 8/88; A61K 8/062; A61Q 19/00; A61K 2800/10

(57) ABSTRACT

A skin external composition having a unique texture and high moisture content, similar to sherbet is disclosed. The skin external composition uses crosslinked polyglutamic acid or its salt. The skin external composition has less stickiness, provides smooth spreadability when applied, and can help maintain the skin moisture content after application.

### CROSS-REFERENCE TO RELATED APPLICATION

This application claims priority under 35 U.S.C. § 119 of Korean Patent Application No. 10-2024-0161678 filed on Nov. 14, 2024, the entire contents of which are incorporated herein by reference.

### BACKGROUND

### (a) Technical Field

One aspect of the present disclosure is a skin external composition that provides a unique texture and high moisture content, similar to a sherbet, using crosslinked polyglutamic acid or its salt.

### (b) Background Art

For products applied to the skin, such as cosmetics, the feeling of use is one of the factors that decisively influences consumer satisfaction, along with the product's functionality or efficacy. Accordingly, products offering various feelings of use have been developed. A representative specific feeling of use for cosmetics is the feeling of bursting water or moisturization, but this feeling of use has the limitation that it can be provided only when the discontinuous phase is the aqueous phase, such as in a water-in-oil type (W/O or W/S, water-in-silicon oil) emulsion. However, these formulations can initially feel strongly greasy because the continuous phase is oil or silicone oil. Additionally, the oil component in the continuous phase may remain on the skin, giving the feeling that only the water component is on the surface, and the oil component or silicone oil component in the continuous phase has the disadvantage of irritating the skin or leaving residue that is difficult to wash off.

Accordingly, there is a need to develop a skin external composition that provides a unique feeling of use and high moisture content without being a water-in-oil type emulsion. [Prior Art Document] [Patent Document] (Patent Document 1) Korean Patent No. 10-2565060

### SUMMARY OF THE DISCLOSURE

One aspect of the present disclosure is to provide a skin external composition having a unique texture and high moisture content, similar to sherbet, using crosslinked polyglutamic acid or its salt.

According to one aspect of the present disclosure, the present disclosure provides a skin external composition of oil-in-water type comprising an aqueous phase and an oil phase, wherein the aqueous phase comprises crosslinked polyglutamic acid or a salt thereof, and the oil phase comprises emulsified particles.

In one embodiment of the present disclosure, the particle size of the crosslinked polyglutamic acid or its salt may be 20 μm to 300 μm.

In one embodiment of the present disclosure, the content of the crosslinked polyglutamic acid or its salt may be 0.001% by weight to 10% by weight based on 100% by weight of the total composition.

In one embodiment of the present disclosure, the particles of the crosslinked polyglutamic acid or its salt may be non-sphere type particles.

In one embodiment of the present disclosure, the composition may have a sherbet texture.

In one embodiment of the present disclosure, the present disclosure provides a method for moisturizing skin, comprising topically applying the composition to the skin.

In one embodiment of the present disclosure, the composition may be a cosmetic composition.

According to another aspect of the present disclosure, the present disclosure provides a method for preparing the skin external composition, comprising the steps of: (1) irradiating an aqueous solution of polyglutamic acid or its salt with an electron beam at a dose of 50 kGy to 300 kGy to form crosslinks between polyglutamic acids; and (2) adjusting the particle size of the crosslinked polyglutamic acid to 20 μm to 300 μm.

In one embodiment of the present disclosure, the weight average molecular weight of the polyglutamic acid or its salt in step (1) above may be 10,000 Da to 80,000 Da.

The skin external composition according to one embodiment of the present disclosure comprises crosslinked polyglutamic acid or its salt, has a unique texture and high moisture content similar to a sherbet, provides a smooth spreadability with little stickiness when applied, and helps maintain skin moisture after application.

### BRIEF DESCRIPTION OF THE DRAWINGS

FIG. 1 shows a reaction mechanism by which polyglutamic acid is crosslinked by irradiation with an electron beam.

FIG. 2 is a graph showing the particle size distribution of Example 1.

FIG. 3 is an optical microscope image showing the particle size of Example 1 (×40 magnification).

FIG. 4 is an optical microscope image showing the particle size of Example 1 (×200 magnification).

FIG. 5 is a photograph of the appearance of Example 1.

FIG. 6 is a photograph of the appearance of Comparative Example 2.

FIG. 7 is a graph showing the results of measuring moisture content over time after application of Example 1 and Comparative Examples 1 and 2.

FIG. 8 is a graph showing the results of evaluating the spreadability of Comparative Example 3.

FIG. 9 is a graph showing the results of evaluating the spreadability of Example 1.

### DETAILED DESCRIPTION

The embodiments provided in accordance with the present disclosure can all be achieved by the following description. It should be understood that the following description describes preferred embodiments of the present disclosure and that the present disclosure is not necessarily limited thereto.

For physical properties described in this specification, if measurement conditions and methods are not specifically described, the physical properties are measured according to measurement conditions and methods generally used by those skilled in the art.

Throughout the present specification, when a part is said to ‘comprise’ a component, this does not mean that it excludes other components, but rather that it may further comprise other components, unless otherwise specifically stated.

Throughout the present specification, “%” means % by weight unless explicitly stated otherwise.

### Skin External Composition

According to one aspect of the present disclosure, the present disclosure provides a skin external composition of oil-in-water type comprising an aqueous and an oil phases, which comprises crosslinked polyglutamic acid or its salt in the aqueous phase and comprises emulsified particles in the oil phase.

The term “skin external composition” as used herein refers to a composition in a formulation that is applied externally to the skin. The skin external composition may be applied directly by hand or using various application tools such as a spatula, and may comprise various formulations of cosmetics, pharmaceuticals, quasi-drugs and the like.

According to one embodiment of the present disclosure, the polyglutamic acid (PGA) may be a water-soluble polymer composed of glutamic acid linked by peptide bonds. Polyglutamic acid has a water retention capacity far superior to hyaluronic acid, but has the disadvantage of being difficult to utilize as a raw material for cosmetics due to its sticky feeling of use. However, by crosslinking polyglutamic acid, stickiness can be reduced and a silky feeling of use can be provided, thereby significantly improving this disadvantage. Additionally, crosslinked polyglutamic acid can retain more moisture than non-crosslinked polyglutamic acid, and its increased stability can enhance resistance to degradation. The term “silky feeling of use” as used herein refers to the feeling of use in which, when applied to the skin, the composition spreads softly and smoothly, giving a silky and soft feel. A composition with a silky feeling of use is non-sticky, moist, and lightly absorbed, allowing it to adhere smoothly to the skin without leaving a residual feeling.

According to one embodiment of the present disclosure, the polyglutamic acid may exist in a salt form, and the salt is not particularly limited, but may be an acid addition salt, a base addition salt, or an amino acid salt. For example, the salt may be inorganic acid salts such as hydrochloride, hydrobromide, sulfate, hydroiodide, nitrate, and phosphate; organic acid salts such as citrate, oxalate, acetate, formate, propionate, benzoate, trifluoroacetate, maleate, tartrate, methanesulfonate, benzenesulfonate, and paratoluenesulfonate; inorganic base salts such as sodium salt, potassium salt, calcium salt, magnesium salt, copper salt, zinc salt, aluminum salt, and ammonium salt; organic base salts such as triethylammonium salt, triethanolammonium salt, pyridinium salt, and diisopropylammonium salt; amino acid salts such as lysine salt, arginine salt, histidine salt, aspartate and glutamate. According to one embodiment of the present disclosure, the salt may be sodium polyglutamate, potassium polyglutamate, polyglutamic acid acetate or ammonium polyglutamate.

According to one embodiment of the present disclosure, the crosslinking of the polyglutamic acid or its salt can be performed by an electron beam, but the crosslinking method is not particularly limited.

According to one embodiment of the present disclosure, the skin external composition may have a texture and appearance similar to a sherbet. In this case, the “sherbet” may refer to a state in which the composition has a particle-like feel and a light and soft foam-like, crumbly physical property when the composition is applied as if pushing it on a glass plate and the appearance is confirmed (see FIG. 5). According to one embodiment of the present disclosure, the composition can be spread smoothly as if melting similar to a sherbet when used, and the moisturizing feeling when the particles of the crosslinked polyglutamic acid or its salt burst and the oily feeling when the emulsified particles burst can be continuously felt.

According to one embodiment of the present disclosure, the composition may comprise, in an aqueous phase, particles of crosslinked polyglutamic acid or its salt having a particle size distribution with a D(0.5) of 20 μm to 300 μm. In this case, the particle size distribution is the ratio of a particle group of a certain particle size range to the entire particle group expressed as a percentage, and the particle size distribution curve is a graphical representation thereof. The D(0.5) is also called D50, D(V, 0.5), or median particle size. The D(0.5) represents the size of the particles distributed in the middle 50% of the particle size distribution diagram, and appears as a peak on the distribution diagram. According to one embodiment of the present disclosure, the above-described 20 μm to 300 μm may refer to the size of at least 50%, 60%, 70%, 80% or 90% of the particles included in the composition. That is, the range may be a value of D(0.5), D(0.6), D(0.7), D(0.8) or D(0.9). According to one embodiment of the present disclosure, the particle size distribution can be measured using a Mastersizer 2000 from Malvern Panalytical company, a particle size analyzer that utilizes laser diffraction. However, as long as it is a particle size analyzer that can measure particle size distribution, it is not limited in its method or type.

According to one embodiment of the present disclosure, the particles of the crosslinked polyglutamic acid or its salt may have a particle size distribution in which D(0.5) is 20 μm to 300 μm, 40 μm to 250 μm, or 60 μm to 200 μm; or 20 μm or more, 40 μm or more, 60 μm or more, 80 μm or more, 100 μm or more, 120 μm or more; and 300 μm or less, 280 μm or less, 260 μm or less, 240 μm or less, 220 μm or less, 200 μm or less. If the D(0.5) value in the particle size distribution is less than 20 μm, the silky feeling of use may not be sufficient, and a sticky residual feeling may be felt due to the use of an excessive amount of thickener. If the D(0.5) value of the gel particles exceeds 300 μm, the particles are excessively large, so that a smooth texture similar to a sherbet does not appear, and a foreign body sensation is felt, which not only reduces the feeling of use but also deteriorates the long-term stability of the formulation.

According to one embodiment of the present disclosure, the particles of the crosslinked polyglutamic acid or its salt may be non-sphere type particles. The “non-sphere type particles” may be particles that exhibit various irregular geometric structures, deviating from the traditional spherical shape, and may have complex structures, such as elongated or flat shapes, or rod-shaped, disc-shaped, ellipsoidal, fibrous, or other asymmetrical shapes, unlike spherical particles having isotropic and uniform curvature. The particles of non-spherical crosslinked polyglutamic acid or its salt according to one embodiment of the present disclosure may be structurally more rigid than other polyglutamic acids having spherical particles, thereby improving the mechanical properties of the composition and providing a unique feeling of use, such as a silky feeling of use. In addition, they may have a larger surface area relative to their volume, thereby providing high moisture content.

According to one embodiment of the present disclosure, the crosslinked polyglutamic acid or its salt may be contained in an amount of 0.001% by weight to 10% by weight based on a total of 100% by weight of the composition. Specifically, the crosslinked polyglutamic acid or its salt may be contained in an amount of 0.001% by weight to 10% by weight, 0.01% by weight to 7% by weight, 0.1% by weight to 4% by weight, and may be contained in an amount of 0.001% by weight or more, 0.01% by weight or more, 0.1% by weight or more, 0.2% by weight or more, 0.3% by weight or more, 0.4% by weight or more, and 10% by weight or less, 9% by weight or less, 8% by weight or less, 7% by weight or less, 6% by weight or less, 5% by weight or less. If it is outside the above range, the unique appearance and texture similar to a sherbet may not appear.

According to one embodiment of the present disclosure, the oil contained in the emulsified particles is not limited in type as long as it is an oil commonly used in the relevant technical field, and may be an ester oil, a hydrocarbon oil, a natural oil, a silicone oil, etc. Specifically, the oil may be at least one selected from the group consisting of polydecene, hydrogenated polydecene, polybutene, hydrogenated polyisobutene, dicaprylyl carbonate, diisostearylmalate, butylene glycol dicaprylate/dicaprate, cetyl 2-ethyl hexanoate, triethylhexanoin, caprylic/capric triglyceride, dicetearyl dimer dilinoleate, diisostearyl malate, hexyl laurate, pentaerythrityl tetraethylhexanoate, pentaerythrityl tetraisostearate, octyldodecyl stearoyl stearate, cyclopentasiloxane, cyclohexasiloxane, dimethicone, methyl trimethicone, cyclomethicone, phenyl trimethicone, squalane, tamanu oil, macadamia nut oil, sunflower seed oil, olive oil, evening primrose oil, argan oil, apricot oil, sesame oil, orange oil, rosewood oil, bergamot oil, camellia oil, tea tree oil and jojoba oil.

According to one embodiment of the present disclosure, the present disclosure provides a method for moisturizing skin, comprising topically applying the composition to the skin.

According to one embodiment of the present disclosure, the skin external composition may be a cosmetic composition. The external form of the cosmetic composition may comprise a cosmetically or dermatologically acceptable medium or base. The cosmetic composition may be provided in any form suitable for topical application, for example, in the form of a solution, a gel, a paste, an anhydrous product, an emulsion obtained by dispersing an oil phase in an aqueous phase, a suspension, a microemulsion, a microcapsule, a microgranule or a vesicle dispersion of ionic (liposomes) and non-ionic type, a cream, a skin lotion, a milky lotion, an ointment, etc. These compositions may be prepared according to conventional methods in the art.

The cosmetic composition is not particularly limited in its formulation and may be formulated as a skin care composition, a makeup composition, a body care composition, a hair care composition, a cleansing composition or the like.

According to one embodiment of the present disclosure, the cosmetic composition can be formulated as cosmetics such as an ampoule, a cream, a softening lotion, an astringent, a nutritional lotion, a nutritional cream, a massage cream, an essence, an eye cream, an eye essence, a cleansing cream, a cleansing foam, a cleansing water, a cleansing tissue containing the cosmetic composition, a pack, a body lotion, a body cream, a body oil, and a body essence. According to one embodiment of the present disclosure, the cosmetic composition may be formulated as cosmetics such as a makeup primer, a makeup base, a foundation, a concealer, a lipstick, a lip gloss, a lip balm, a powder, a lip liner, an eyeliner, a mascara, an eyebrow, an eye shadow, a blusher, a twin cake, a sunscreen or the like. According to one embodiment of the present disclosure, the cosmetic composition may be formulated as a body wash, a facial wash, a hand wash, a hair shampoo, a hair rinse, a hair conditioner, a hair treatment, a hair tonic, a scalp treatment, a scalp and hair mixed-use treatment, a hair lotion, a hair cream, a hair nutritional lotion, a general ointment or the like.

According to one embodiment of the present disclosure, when the formulation is a paste, a cream or a gel, animal fiber, plant fiber, wax, paraffin, starch, tragacanth, cellulose derivatives, polyethylene glycol, silicone, bentonite, silica, talc, zinc oxide or the like can be used as a carrier component. According to one embodiment of the present disclosure, when the formulation is a solution or an emulsion, a solvent, a solvating agent or an emulsifying agent may be used as a carrier component, for example, water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan may be used. According to one embodiment of the present disclosure, when the formulation is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth or the like can be used as a carrier component. According to one embodiment of the present disclosure, when the formulation is a surfactant-containing cleansing agent, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glyceride, fatty acid diethanolamide, vegetable oil, lanoline derivative, ethoxylated glycerol fatty acid ester or the like may be used as a carrier component.

According to one embodiment of the present disclosure, the cosmetic composition may further include functional additives and ingredients included in general cosmetic compositions. The functional additives may include ingredients selected from the group consisting of water-soluble vitamins, oil-soluble vitamins, high molecular peptides, high molecular polysaccharides, sphingolipids, and seaweed extracts. In addition, the compounding ingredients may include maintenance ingredients, moisturizers, emollients, surfactants, organic and inorganic pigments, organic powders, ultraviolet absorbers, preservatives, bactericides, antioxidants, plant extracts, pH adjusters, alcohol, pigments, fragrances, blood circulation promoters, cooling agents, antiperspirants, purified water, etc.

According to one embodiment of the present disclosure, the composition may be a pharmaceutical composition. The pharmaceutical composition may be formulated as a parenteral dosage form in a semi-solid or liquid form by adding a commonly used inorganic or organic carrier to the active ingredient used in one embodiment of the present disclosure. The composition according to one embodiment of the present disclosure can be easily formulated with an active ingredient by a conventional method widely known in the art, and in this case, surfactants, excipients, colorants, flavorings, preservatives, stabilizers, buffers, suspending agents, and other commonly used auxiliary agents can be appropriately used. The dosage of the active ingredient of the pharmaceutical composition according to one embodiment of the present disclosure will vary depending on the age, sex, body weight, pathological condition and its severity of the subject to be administered, the route of administration, or the prescriber's judgment. Determination of an appropriate dosage based on these factors is within the level of a person skilled in the art, and the daily dosage may be, for example, but is not limited to, 0.1 mg/kg/day to 100 mg/kg/day, more specifically, 5 mg/kg/day to 50 mg/kg/day.

### Method for Producing Skin External Composition

According to one aspect of the present disclosure, the present disclosure provides a method for preparing the skin external composition described above. According to one embodiment of the present disclosure, the method for preparing the skin external composition comprises the steps of: (1) irradiating an aqueous solution of polyglutamic acid or its salt with an electron beam at a dose of 50 kGy to 300 kGy to form crosslinks between polyglutamic acids; and (2) adjusting the particle size of the crosslinked polyglutamic acid to 20 μm to 300 μm.

Since the specific description of polyglutamic acid or its salt according to the method of preparing the skin external composition is as described above, the specific description will be omitted below.

In step (1) above, an electron beam is irradiated to an aqueous solution of polyglutamic acid or its salt at a dose of 50 kGy to 300 kGy to form crosslinks between polyglutamic acids.

According to one embodiment of the present disclosure, the formation of crosslinks between the polyglutamic acids can be achieved chemically by a crosslinking agent or by irradiating with an electron beam, but is not limited thereto as long as it is a method capable of forming crosslinks.

According to one embodiment of the present disclosure, when the crosslinking between the polyglutamic acids is achieved by irradiation with an electron beam, the irradiation dose of the electron beam may be 50 kGy to 300 kGy. Specifically, the irradiation dose of the electron beam may be 50 kGy to 300 kGy, 80 kGy to 250 kGy, or 110 kGy to 200 kGy, and may be 50 kGy or more, 60 kGy or more, 70 kGy or more, 80 kGy or more, or 90 kGy or more, and 300 kGy or less, 270 kGy or less, 240 kGy or less, 210 kGy or less, or 180 kGy or less. when the above-described range is satisfied, the crosslinking between polyglutamic acids can sufficiently progress, so that the composition can have a unique texture similar to a sherbet and excellent spreadability.

According to one embodiment of the present disclosure, the weight average molecular weight of the polyglutamic acid or its salt may vary depending on the length of the chain and may be from 10,000 Da to 80,000 Da. Specifically, the weight average molecular weight of the polyglutamic acid or its salt may be 10,000 Da to 80,000 Da, 20,000 Da to 70,000 Da, or 30,000 Da to 60,000 Da, and may be 10,000 Da or more, 15,000 Da or more, 20,000 Da or more, 25,000 Da or more, or 30,000 Da or more, and 80,000 Da or less, 75,000 Da or less, 70,000 Da or less, 65,000 Da or less, or 60,000 Da less. When the above-described range is satisfied, the composition can contain sufficient moisture and exhibit a unique appearance and texture similar to a sherbet.

In step (2) above, the particle size of the crosslinked polyglutamic acid is adjusted to 20 μm to 300 μm.

According to one embodiment of the present disclosure, the method is not limited as long as it can be manufactured into a particle size within the size range described above, and it can be manufactured by passing the crosslinked polyglutamic acid through a mesh to be pulverized, and the particle size can also be adjusted by stirring with a homo mixer.

According to one embodiment of the present disclosure, when the particle size is adjusted using a homo mixer, the particle size can be adjusted by stirring the crosslinked polyglutamic acid at 5,000 rpm to 15,000 rpm for 1 to 10 minutes.

According to one embodiment of the present disclosure, the skin external composition can be prepared by adding and mixing the crosslinked polyglutamic acid into an oil-in-water composition prepared according to a conventional method for preparing an oil-in-water composition in the relevant industry. According to one embodiment of the present disclosure, the method for preparing the conventional oil-in-water composition may be a method of preparing an aqueous phase portion containing an aqueous component and an oil phase portion containing an oily component, and then adding an oil phase portion to the aqueous phase portion and stirring them, but is not limited thereto.

Hereinafter, preferred examples are presented to help understand the present disclosure, but the following examples are provided only to make it easier to understand the present disclosure, and the present disclosure is not limited thereto.

### PREPARATION EXAMPLES

### Example 1 and Example 4 (Cream Type)

The composition of Example 1 and Example 4, having the composition shown in Table 1 below, was prepared by the following method.

First, crosslinked polyglutamic acid (Manufacturer: Hyundai Bioland) was irradiated with an electron beam of 140 kGy, and stirred at 9,000 rpm for 3 minutes using a homo mixer (Manufacturer: Primix, Product name: Mark2 2.5) to produce particles with D(0.5) ranging from 20 μm to 300 μm in the particle size distribution.

Then, the crosslinked polyglutamic acid was added and mixed into the oil-in-water composition prepared according to the composition of Table 1 below to prepare the composition of Example 1 and Example 4. Specifically, dicaprylyl ether, squalane, and C14-22 alcohol*C12-20 alkyl glucoside were added to the reactor in the composition ratios shown in Table 1, and the mixture was sufficiently heated to 75° C. using an AGI mixer (Manufacturer: Shinwon Machinery, Model: AGI TATOR (2)) to completely dissolve the oil phase portion. Separately, sodium metaphosphate and propanediol were added to distilled water in the composition ratios shown in Table 1, and the mixture was heated to 75° C. to completely dissolve the aqueous phase portion. While slowly adding the oil phase portion to the aqueous phase portion, the mixture was stirred at high speed at 9000 rpm for 3 minutes using a homo mixer (Manufacturer: Primix, Model: Mark2 2.5). The mixture was stirred at 9000 rpm for 2 minutes while slowly adding polyacrylate-13 to the mixture, and then the crosslinked polyglutamic acid was added and stirred at 9000 rpm for another 2 minutes to prepare a skin external composition.

### Example 2 (Toner Type)

The composition of Example 2, having the composition shown in Table 1 below, was prepared by the following method.

Sodium metaphosphate and propanediol were added to distilled water in the composition ratios shown in Table 1 in a reactor, and then the aqueous phase portion was completely dissolved at room temperature. Crosslinked polyglutamic acid prepared in the same manner as Example 1 was added to the aqueous phase portion and stirred at 9000 rpm for 2 minutes to prepare a skin external composition.

### Example 3 (Emulsion Type)

The composition of Example 3, having the composition shown in Table 1 below, was prepared by the following method.

Dicaprylyl ether, squalane, and C14-22 alcohol*C12-20 alkyl glucoside were added to the reactor in the composition ratios shown in Table 1, and the mixture was sufficiently heated to 75° C. using an AGI mixer (Manufacturer: Shinwon Machinery, Model: AGI TATOR (28)) to completely dissolve the oil phase portion. Separately, sodium metaphosphate and propanediol were added to distilled water in the composition ratios shown in Table 1, and the mixture was heated to 75° C. to completely dissolve the aqueous phase portion. While slowly adding the oil phase portion to the aqueous phase portion, the mixture was stirred at high speed at 9000 rpm for 3 minutes using a homo mixer (Manufacturer: Primix, Model: Mark2 2.5). The mixture was stirred at 9000 rpm for 2 minutes while slowly adding polyacrylate-13 to the mixture, and then the crosslinked polyglutamic acid was added and stirred at 9000 rpm for another 2 minutes to prepare a skin external composition.

### Comparative Example 1

A skin external composition was prepared in the same manner as in Example 1 according to the composition in Table 1 below, except that instead of crosslinked polyglutamic acid with D(0.5) of 20 μm to 300 μm, crosslinked polyglutamic acid with a D(0.5) exceeding 300 μm due to not stirring the crosslinked polyglutamic acid using a homo mixer was added.

### Comparative Example 2

A skin external composition was prepared in the same manner as in Example 1, according to the composition in Table 1 below, except that non-crosslinked polyglutamic acid was added instead of crosslinked polyglutamic acid with D(0.5) of 20 μm to 300 μm.

### Comparative Example 3

A skin external composition was prepared in the same manner as in Example 1 according to the composition in Table 1 below, except that crosslinked polyglutamic acid with D(0.5) of 20 μm to 300 μm was not added.

TABLE 1
(Unit: % by weight)

| | | | | | Comparative | Comparative | Comparative |
|---|---|---|---|---|---|---|---|
| | Example 1 | Example 2 | Example 3 | Example 4 | Example 1 | Example 2 | Example 3 |
| Dicaprylyl Ether | 4 | 0 | 2 | 4 | 4 | 4 | 4 |
| Squalane | 2 | 0 | 2 | 2 | 2 | 2 | 2 |
| C14-22 | 2 | 0 | 1 | 2 | 2 | 2 | 2 |
| alcohols*C12-20 | | | | | | | |
| alkyl glucoside | | | | | | | |
| Sodium | 0.02 | 0.02 | 0.02 | 0.02 | 0.02 | 0.02 | 0.02 |
| Metaphosphate | | | | | | | |
| Propanediol | 8 | 8 | 8 | 8 | 8 | 8 | 8 |
| Polyacrylate-13 | 1 | 0 | 0.5 | 1 | 1 | 1 | 1.5 |
| Crosslinked | 0.4 | 0.6 | 0.4 | 1.2 | 0 | 0 | 0 |
| Polyglutamic Acid | | | | | | | |
| (20-300 μm) | | | | | | | |
| Crosslinked | 0 | 0 | 0 | 0 | 0.4 | 0 | 0 |
| Polyglutamic Acid | | | | | | | |
| (exceeding 300 μm) | | | | | | | |
| Polyglutamic Acid | 0 | 0 | 0 | 0 | 0 | 0.4 | 0 |
| Distilled water | To 100 | To 100 | To 100 | To 100 | To 100 | To 100 | To 100 |

### EXPERIMENTAL EXAMPLES

### Experimental Example 1 (Evaluation of pH and Viscosity)

The pH, hardness and viscosity of the skin external compositions of Example 1 to 4 and Comparative Examples 1 to 3 were measured. Viscosity was measured using a rotational viscometer (RVDV2T, BROOKFIELD), hardness was measured using a hardness meter (CR-100, Sun Scientific), and pH was measured using a pH meter (S-220 Mettler, Mettler Toledo), all at room temperature (26° C.). The results of the measurements are summarized in Table 2 below.

TABLE 2

| | pH | Hardness (N) | Viscosity (cps) |
|---|---|---|---|
| Example 1 | 7.0 | 12 | — |
| Example 2 | 7.0 | — | 1,000 |
| Example 3 | 7.0 | — | 10,000 |
| Example 4 | 7.0 | 20 | |
| Comparative Example 1 | 7.0 | 10 | — |
| Comparative Example 2 | 3.8 | — | 25,000 |
| Comparative Example 3 | 6.4 | 13 | — |

Cosmetics can be distributed only when the pH is measured to be 3.0 to 9.0. Example 1 to 4 and Comparative Examples 1 to 3 were all measured to satisfy the above-described range, and the hardness of Example 1, Comparative Example 1, and Comparative Example 3 were measured to be similar. In addition, the viscosity of Example 2 and Example 3 was measured to be lower than that of Comparative Example 2.

### Experimental Example 2 (Evaluation of Particle Size of Skin External Composition)

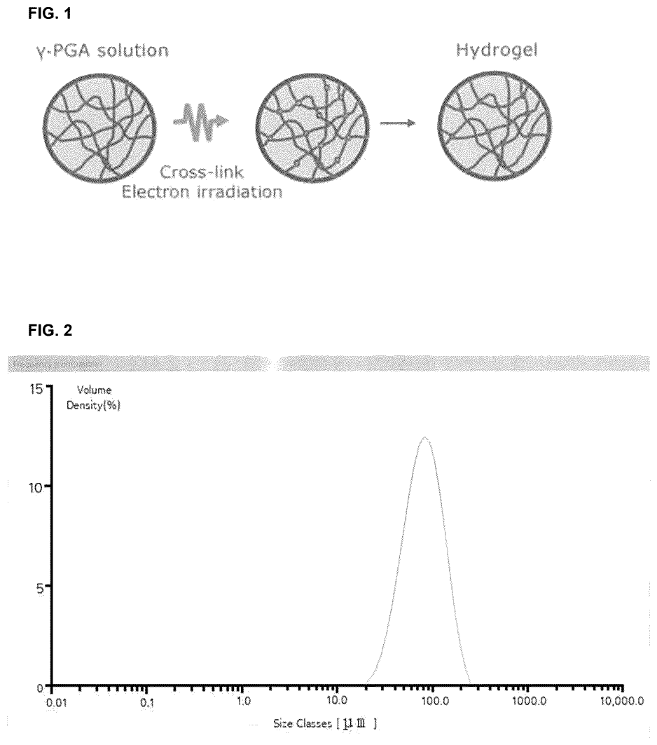

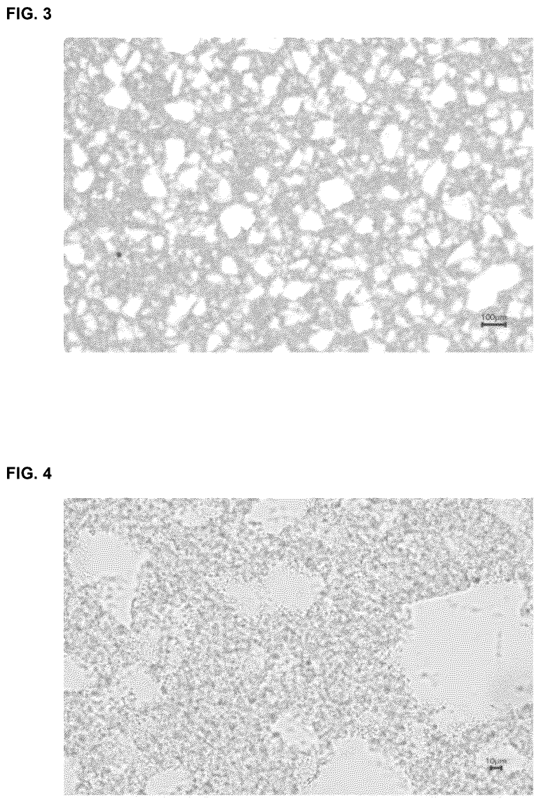

The particle size distribution of the skin external composition of Example 1 was measured using a Mastersizer 2000 from Malvern Panalytical company, and the results are shown in FIG. 2. In addition, the particle size of the skin external composition of Example 1 was measured using an optical microscope (Manufacturer: Nikon Instruments Korea Co., Ltd., Model: Microscope 80i, ×40×, ×200×), and the results are shown in FIGS. 3 and 4, respectively.

It was found that according to FIG. 2, Example 1 shows a peak (D(0.5)) of emulsified particles in the range of 20 μm to 300 μm, and according to FIGS. 3 and 4, Example 1 has a particle size of about 20 μm to 300 μm and a particle shape of a non-sphere type.

### Experimental Example 3 (Evaluation of the Appearance and Feeling of Use of Skin External Composition)

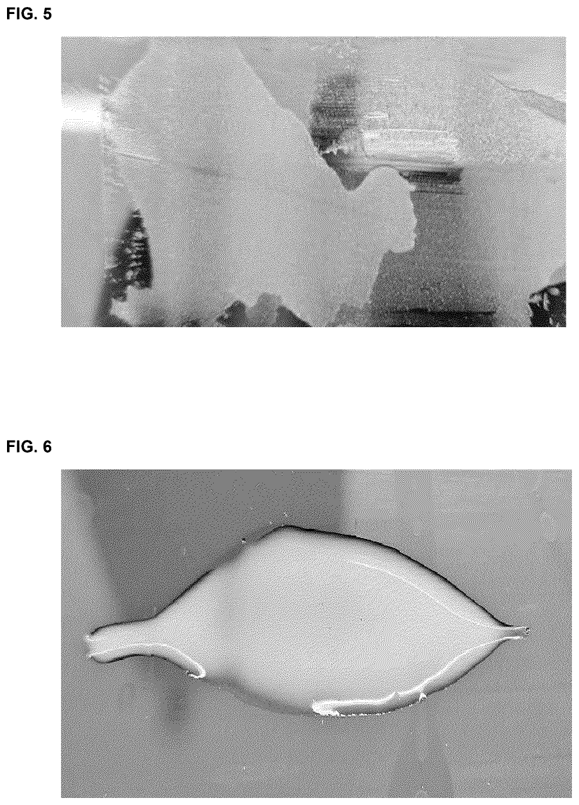

The skin external compositions of Example 1 and Comparative Example 2 were thinly applied to a glass plate as if pushing it, and then photographed, and the results are shown in FIGS. 5 and 6. In addition, about 0.3 ml of each of the skin external compositions of Example 1 and Comparative Examples 1 to 3 were taken and placed on the back of the hand, and the overall feeling of use felt on the back of the hand was confirmed when applied for about 20 seconds under the condition of rolling twice per second.

According to FIGS. 5 and 6, Example 1 containing crosslinked polyglutamic acid exhibited a unique appearance of sherbet texture, whereas Comparative Example 2 containing non-crosslinked polyglutamic acid exhibited a simple appearance of emulsion.

Example 1 exhibited a silky feel of use, whereas Comparative Example 1 exhibited poor formulation stability over time and felt like a foreign substance, like a scrub, and Comparative Example 2 felt strongly sticky when used, and the viscosity of the contents was low at the same thickener content. Comparative Example 3 also lacked a flexible feel when applied, and the sticky residual feeling increased due to the increased amount of thickener to achieve a viscosity similar to Example 1.

### Experimental Example 4 (Evaluation of Moisture Content of Skin External Crosslinked Gel)

The skin moisture content was measured using the skin external compositions of Example 1 and Comparative Examples 1 and 2. Specifically, the skin moisture content was measured using Corneometer CM825 WL immediately before applying the skin external composition to the forearm of an adult male, 3 hours after application, and 6 hours after application. The results are shown in FIG. 7.

According to FIG. 7, it can be seen that the composition of Example 1 maintains an overall higher moisture content compared to Comparative Examples 1 and 2.

### Experimental Example 5 (Evaluation of Spreadability of Skin External Crosslinked Gel)

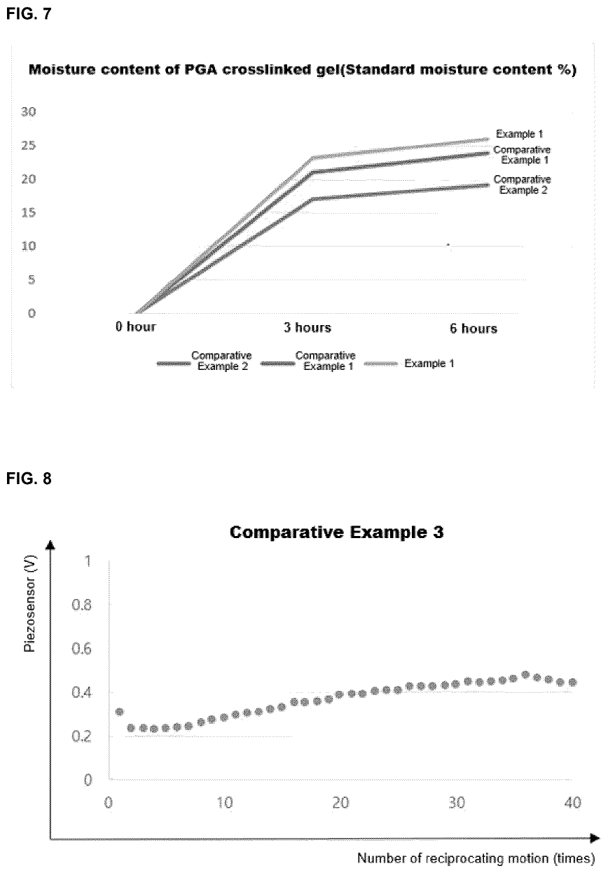

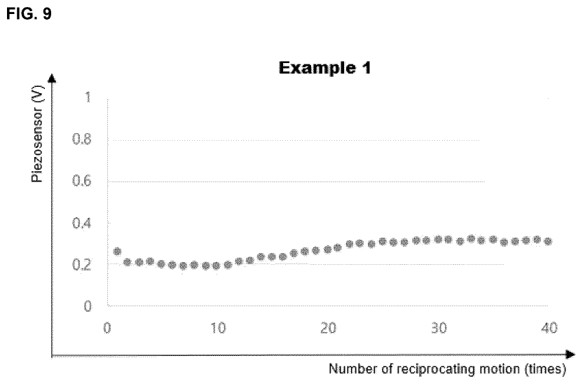

The spreadability of the skin external compositions of Example 1 and Comparative Example 3 was measured. Equal amounts of each composition were applied to artificial leather, and the V value was measured using a device (Measuring device: UTPSM (Universal Tactile Pressure Sensor Measurement) System-based development device, brand name: Sensornoid, Terra Reader Co., Ltd., Korea) that quantifies friction force and adhesion as a Piezosensor coefficient of friction. Specifically, under the condition where the sample was applied to the artificial leather, a fingerprint pattern PDMS sensor was applied vertically with a constant pressure (0.02 N). Subsequently, the sensor was moved horizontally at a speed of 7 mm/s, and the friction force was continuously measured in units of 1/100 second. The results are shown in FIGS. 8 and 9. The lower the V value, the lower the resistance of the contents and the better the spreadability of the contents.

According to FIGS. 8 and 9, it can be seen that Example 1 has a V value less than 0.4 throughout the entire range, thereby imparting a rolling sensation that allows the contents to be smoothly applied to the skin, and it can be seen that Comparative Example 3 has a section where the V value exceeds 0.4, and thus has higher resistance and poorer spreadability than Example 1.

From the results described above, it can be seen that the skin external composition of the present disclosure has a silky feeling of use and excellent spreadability, and also secures excellent water retention capacity.

All simple modifications or changes of the present disclosure fall within the scope of the present disclosure, and the specific scope of protection of the present disclosure will be made clear by the appended claims.

What is claimed is:

1. A skin external composition of oil-in-water type comprising an aqueous phase and an oil phase, wherein the aqueous phase comprises crosslinked polyglutamic acid or its salt, and the oil phase comprises emulsified particles.

2. The skin external composition according to claim 1, wherein the particle size of the crosslinked polyglutamic acid or its salt is 20 μm to 300 μm.

3. The skin external composition according to claim 1, wherein the content of the crosslinked polyglutamic acid or its salt is from 0.001% by weight to 10% by weight based on a total of 100% by weight of the composition.

4. The skin external composition according to claim 1, wherein the particles of the crosslinked polyglutamic acid or its salt are non-sphere type particles.

5. The skin external composition according to claim 1, wherein the composition has a sherbet texture.

6. A method for moisturizing skin, comprising: topically applying to the skin the composition of claim 1.

7. The skin external composition according to claim 1, wherein the composition is a cosmetic composition.

8. A method for preparing the skin external composition of claim 1, comprising the steps of:
(1) irradiating an aqueous solution of polyglutamic acid or its salt with an electron beam at a dose of 50 kGy to 300 kGy to form crosslinks between polyglutamic acids; and
(2) adjusting the particle size of the crosslinked polyglutamic acid to 20 μm to 300 μm.

9. The method for preparing the skin external composition according to claim 8, wherein the weight average molecular weight of polyglutamic acid or its salt in step (1) above is 10,000 Da to 80,000 Da.
